(19) United States
(12) Patent Application Publication
(10) Pub. No.: US 2021/0223421 A1
(43) Pub. Date: Jul. 22, 2021

(54) COMPOSITE MATERIAL FOR DETECTING FREE NEUTRONS WITH AN EFFECTIVE ATOMIC NUMBER SIMILAR TO BODY TISSUE BY USING BERYLLIUM OXIDE AND/OR LITHIUM TETRABORATE, DOSIMETER, AND A METHOD FOR CAPTURING OR DETECTING FREE NEUTRONS

(71) Applicant: Dosimetrics GmbH, Munich (DE)

(72) Inventor: Peter Georg SCHEUBERT, Miesbach (DE)

(21) Appl. No.: 17/123,423

(22) Filed: Dec. 16, 2020

(30) Foreign Application Priority Data
Dec. 20, 2019 (EP) 19218975.1

(51) Int. Cl. G01T 3/06; A61N 5/10

(52) U.S. Cl. CPC G01T 3/06; A61N 2005/109; A61N 5/1071

(57) ABSTRACT

A method as well as a composite material for detecting free neutrons are disclosed that include a converter material, which is configured to generate in response to a capture of neutrons a secondary radiation, and a detector material, which is configured to store an information relating to the secondary radiation and to release it again in a later evaluation by optically stimulated luminance. The converter material and the detector material each are present in a plurality of particles, which are jointly present in the composite material as material mixture. In order to improve the detection of neutrons with regard to a person dosimetry, that is the estimation of a dose absorbed by a human, it is envisaged that the detector material is formed from beryllium oxide and/or the converter material is formed from lithium tetraborate.

The present invention relates to a composite material for detecting free neutrons with a converter material, which is configured to generate in response to a capture of neutrons a secondary radiation, and a detector material, which is configured to store an information relating to a quantity of the secondary radiation, and to release it again in a later evaluation by optically stimulated luminescence, wherein the converter material and the detector material each consist of a plurality of particles, which jointly are present in the composite material as material mixture. Moreover, the invention relates to a dosimeter, which comprises such composite material. Finally, the invention also relates to respective methods for capturing or for detecting free neutrons by a composite material.

For measuring or detecting radiation, in particular ionizing radiation, various types of dosimeters are known. For measuring devices for establishing cumulated radiation doses the designation dosimeter is common. A low-cost realization variant nevertheless ensuring high accuracy is the employment of passive dosimeters. Passive dosimeters are characterized in that the radiation energy absorbed by the dosimeter in a time-invariant and permanent manner is stored in the structure of the detector material. The free radiation carriers generated by the ionizing radiation in the detector may partially serve directly as information storage for the information. In particular in the detector material free electrons generated by ionizing processes can reach energy levels which on the one hand have a raised energy level in comparison with the basic state but cannot reach the basic state or raised excitation states at room temperature. These electrons thus are proverbially trapped at such energy level, the English term for corresponding energy levels being traps.

For a long time, it was common to equip such passive dosimeters with a detector material which can be evaluated according to the principle of thermoluminescence dosimetry (TLD). During readout, the electrons are freed by thermal stimulation (heating the detector material) from the energy levels or traps, in which they were trapped. Hereby the corresponding electrons from the traps typically reach higher energies in the conduction band of the detector material. In a subsequent recombination with the associated holes light of a specific wavelength is emitted. From the intensity of the light or the photon number of the light a conclusion may be drawn as to the cumulated radiation exposition of the detector and thus the absorbed radiation dose. In particular, in the case of suitable detector materials the intensity or photon number, respectively, of the luminescence light over large portions is proportional to the absorbed radiation dose.

As modern method for the person dosimetry optically stimulating luminescence (OSL) has grown increasingly accepted. The storage of the dosage information is affected analogously to TLD equally in stable traps, that is energy levels between valence and conduction band of the detector material. The release of the trapped electrons, which in a way analogous to the TL process requires the supply of energy, herein, however, is affected by an illumination of the corresponding detector material with light. An advantage of using optically stimulated luminescence is the fact that light power may be used for an immediate and pulseable energy supply, whereas the heating of the detector requires time and possibly expensive devices in order to be able to guarantee a defined heating process. Further, materials suitable for OSL dosimetry are characterized by time invariance of the stored information.

A process leading to the loss of stored information is referred to as fading. In materials in which fading takes place already at room temperature electrons may fall from the energy level of their trap back to a normal energy level and no longer are available as information carriers. From this results a distortion of the measuring result and thus major, time-dependent errors in the subsequent determining of the radiation dose.

Frequently, also the measurement of neutron radiation is of interest. Detector materials which work according to the principle of the optically stimulated luminescence frequently do not respond to the neutron radiation because the isotopes present in the detector comprise no significantly large capture cross sections for neutrons and therefore do not directly interact with the radiation field. So-called converter materials, which are capable of capturing neutrons, provide relief. Typically, a neutron capture timely follows the radioactive decay of the generated isotope, that is the converter materials emit secondary radiation. This secondary radiation in turn is ionizing and can be captured by the detector material.

From the US 2013/0015339 A1 for instance a device to measure the radiation in wells of geological formations is known. A sensing arrangement of the device therein comprises a material facilitating evaluation by optically stimulated luminescence. The sensing arrangement further may comprise a converting layer so as to convert non-ionizing radiation, for instance neutrons, into ionizing radiation.

The publication “Development of new optically stimulated luminescence (OSL) neutron dosimeters” by E. G. Yukihara et. al. suggests the mixing with aluminum oxide (AL₂O₃) as detector material and a converter material for capturing neutrons.

It is the object of the present invention to improve detection of neutrons with regard to the application in person dosimetry, that is the estimation of a dose absorbed by a human.

This object according to the invention is solved by the subject matters of the independent patent claims. Advantageous embodiments and expedient further developments of the invention are subject matter of the subclaims.

The present invention is based on the idea that an effective atomic number of the detector material used in the dosimeter may also have major influence on the neutron dosimetry. It is true that neutrons are only influenced to a lesser degree by the nuclear charge of atoms, however, already here the accuracy of a dose determination can be improved, the closer the effective atomic number of a composite material of converter material and detector material used for passive dosimetry is to the effective atomic number of human tissue. Further advantages, moreover, result if simultaneously with a neutron dose also a dose of ionizing radiation is to be determined, which typically occurs simultaneously with the neutrons. The determination of a reference value for ionizing radiation e.g., gamma radiation, is necessary since neutron dosimeters are intended to quantify the pure neutron share of the radiation, that is a share of other types of radiation occurring at the same time needs to be compensated for. The precise determination of the neutron dose therefore commonly requires the establishing of a reference value for simultaneously incident photon radiation, which subsequently is subtracted to determine the neutron dose. By a raised accuracy of the reference value therefore also the accuracy in determining the neutron dose can be improved.

Starting from these basic considerations, the invention provides a composite material for detecting free neutrons which in terms of its effective atomic number compared with established TL or OSL detector materials is improved and adapted to the atomic number of the human tissue.

The types of radiation for the detection of which the composite material should be directly usable or for the capture of which the converter material is configured are in particular thermal neutrons. Alternatively, or additionally, also fast neutrons can be detected. In particular the composite material is configured for the detection of neutron radiation. Since capture cross sections for neutrons drastically decrease with increasing energy, for the detection of fast neutrons it may be reasonable to reduce their velocity prior to entering the detector material, that is to moderate them. For detecting fast neutrons, a dosimeter, in which the composite material is employed, in addition to the composite material may comprise a moderator material. Alternatively, or additionally, the composite material may be expanded by such moderator material. Alternatively, or additionally, it is possible that the converter material and/or the detector material act as moderator material. The moderator material is characterized in that it is suitable for slowing fast neutrons down. In other words, fast neutrons are reduced by the moderator material in their kinetic energy. By the reduction of the kinetic energy or by slowing the fast neutrons down these can be transferred into a state in which these can be captured with a larger capture cross section. Thermal neutrons are free neutrons with a kinetic energy of about 25 millielectronvolt. It may be envisaged that the converter material besides thermal neutrons also captures fast neutrons with sufficient capture cross section. In this case it could be done without the moderator material for slowing down fast neutrons. For many applications, the detection of fast neutrons is irrelevant, for such applications a moderator material may be done without and it is moreover not necessary that the converter material can capture fast neutrons with large capture cross section.

As already mentioned initially, the converter material is configured to capture neutrons and to generate a secondary radiation in response to such a capturing. The secondary radiation is in particular a different type of radiation than the neutron radiation. For instance, the secondary radiation is an ionizing radiation, for instance photons, in particular x-ray or gamma radiation, or high-energy particles such as beta radiation, alpha radiation, tritium radiation, or the like. Generally, the secondary radiation may preferably be ionizing radiation. In any case the converter material is to be chosen in such a way that same in response to the capture of the neutrons generates a suitable secondary radiation, which can be quantified by the detector material.

The detector material is configured to store in response to a capture of the secondary radiation an information relating to the secondary radiation. The evaluation of the information is affected in particular by the principle of optically stimulating luminescence (OSL). Its principle was already initially set out and therefore is not newly described here. Thus, the detector material is configured to store information by a process, which is accessible to later evaluation by the principle of optically stimulating luminescence (OSL). For instance, the detector material is configured for storing the information in response to an absorption, a scattering, or an (inelastic) collision with a photon, electron, tritium nucleus, or helium nucleus of the secondary radiation. The penetration with secondary radiation is in particular dose-proportional relative to the number of captured free neutrons. Advantageously, it is envisaged that the detector material is configured to release the stored information in a later evaluation dose-proportionately in the form of an emission spectrum or of luminescence light.

In the course of the later evaluation the detector material is illuminated, and thus stimulated, in particular with a stimulation spectrum, in particular monochromatic light of a certain wavelength. As a consequence of the illumination or stimulation the detector material emits the stored information by an emission spectrum. In this connection the typically clearly more intense stimulation light of the stimulation spectrum needs to be separated by suitable optical filters from the, in the case of low radiation doses, very weak luminescence light of the emission spectrum, which is possible only due to a difference in wavelength. In the case of a mixture of different detector materials it is to be reckoned with the emission of the luminescence light being affected at different wavelengths. This means that on the basis of the response signal and its wavelength or by the choice of different stimulation spectra, in particular with monochromatic light of a different wavelength, a separate or sequential evaluation of the different detector materials may be possible.

It is advantageous to design the detector unit as composite material, that is from a material mixture consisting in each case of finely split converter material and detector material. In the material mixture the converter material and the detector material at least substantially are fully mixed or stirred. It is to be ensured that close to each particle with converter material in immediate vicinity there are particles with detector material. In this way a sensitivity of the composite material can be maximized. The secondary radiation only needs to cover an as short as possible distance from the converter material to the detector material. In this way an undesired alternative absorption of the secondary radiation, e.g., in the converter material itself, is limited.

For solving the above-named object or for adapting the effective atomic number of the composite material to the effective atomic number of human tissue, respectively, it is envisaged according to the invention to form the detector material and/or the converter material from a respective material, which in each case has a corresponding effective atomic number. According to the invention this is the case for beryllium oxide (BeO) as well as lithium tetraborate (Li₂B₄O₇). Beryllium oxide in this connection lends itself as detector material, whereas lithium tetraborate is suited as converter material, if the lithium atoms at least partially, in particular at a significant percentage at least 5%, at least 10%, at least 20%, or at least 30%, are formed from the isotope ⁶Li. Additionally lithium tetraborate is also suitable as detector material.

A first aspect of the present invention thus relates to a composite material for detecting free neutrons, comprising a converter material, which is configured to generate in response to a neutron capture a secondary radiation, and a detector material, which is configured to store an information relating to a quantity of the secondary radiation and in a later evaluation to release same again by optically stimulated luminescence, wherein the converter material and the detector material each are present in a plurality of particles, which are jointly present in the composite material as material mixture. Inventive for the first aspect of the present invention is the fact that the detector material is formed from beryllium oxide.

A second aspect of the present invention relates to a composite material for detecting free neutrons, comprising a converter material, which is configured to generate as a consequence of a neutron capture a secondary radiation, and a detector material which is configured to store an information relating to a quantity of the secondary radiation and in a later evaluation to release same again by optically stimulated luminescence, wherein the converter material and the detector material each are present in a plurality of particles, which jointly are present in the composite material as material mixture. Inventive for the second aspect of the present invention is the fact that the converter material is formed from lithium tetraborate.

The respective composite material in its composition is not limited to the converter material and the detector material. The composite material may additionally comprise further materials, such as for instance above-named moderator material or binding agents. As initially described, by way of approximation of the effective atomic number of the composite material in the direction of the effective atomic number of human tissue the efficiency and/or the accuracy and/or reliability of the composite material can be improved as part of the person dosimetry. Since the named materials beryllium oxide and lithium tetraborate each as such already involve the equality of the effective atomic number of the body tissue, these are equally suited for solving the named object by the same inventive idea.

According to a further development of the composite material according to the second aspect it is envisaged that in the lithium tetraborate the isotopes ⁶Li and/or ¹⁰B compared with their natural frequency are enriched. In other words, in the lithium contained in the lithium tetraborate the lithium isotope with the nucleon number 6 may be enriched compared to the natural frequency of the lithium isotope. Alternatively, or additionally, in the boron contained in the lithium tetraborate the boron isotope may be enriched with the nucleon number 10 compared with the natural frequency of the boron isotope. The isotopes ⁶Li and ¹⁰B each have a clearly larger capture cross section for neutrons than the remaining isotopes of the respective element. Thus, by a corresponding enrichment the capture cross section of the lithium tetraborate for neutrons can be raised or improved, respectively. In this way a larger share of the free neutrons incident upon the converter material can be captured. As a consequence, hereby the quantity of the generated secondary radiation is raised since their emission is affected proportionately to the capture of neutrons. On the whole, an effectivity and also an accuracy of the composite material for detecting neutrons can thus be improved.

According to a further development it is envisaged that in the case of the composite material according to the second aspect of the present invention the detector material is formed from a different material than lithium tetraborate. In other words, the corresponding composite material according to this embodiment in addition to the lithium tetraborate, which forms the converter material, comprises a further material as detector material. Thus, a detector material having correspondingly favorable properties for this purpose can be chosen.

According to a further development of the composite material according to the second aspect of the present invention it is envisaged that the detector material is equally formed from lithium tetraborate. In other words, the lithium tetraborate forms both the detector material as well as the converter material of the composite material. This is due to the fact that also lithium tetraborate is suited for the method of the optically stimulated luminescence. In this way by the lithium tetraborate both the objects of the converter material and of the detector material can be executed and the problem of an effective mixing of two different materials is rendered moot.

With regard to the afore-mentioned further development, according to which the detector material is formed from a different material than lithium tetraborate, it becomes evident against this background that in this case two materials which are usable as detector materials form part of the composite material. In other words, the composite material on the one hand contains the lithium tetraborate, which is also usable as detector material, and additionally the other material, which correspondingly is equally usable as detector material. In this way the composite material is accessible to a two-step evaluation by optically stimulated luminescence. Particularly advantageously, the other material, which forms the detector material is chosen in such a way that its emission spectrum can be separated from the emission spectrum of the lithium tetraborate. In analogy the other material, which forms the detector material, can have an emission spectrum, which is specific to the other material, which differs from the emission spectrum that is specific to the lithium tetraborate. In this way the composite material is suited for a double evaluation by optically stimulated luminance with different stimulation spectra and emission spectra in each case. The plural evaluability commonly results in a raised accuracy of the measurement.

According to a further development of the composite material according to the first aspect of the invention and the second aspect of the invention it is envisaged that the detector material is formed of beryllium oxide and the converter material from lithium tetraborate. In other words, the composite material according to this further development comprises beryllium oxide as detector material and lithium tetraborate as converter material. It is a matter of course that the further developments, which with regard to the use of lithium tetraborate are implemented as converter material and/or detector material, equally apply in analogy to this further development. In particular also in the case of a use of beryllium oxide as detector material and lithium tetraborate as converter material it may be envisaged that the isotopes ⁶Li and/or ¹⁰B in the lithium tetraborate compared to their natural frequency are enriched. Also, for the present combination of lithium tetraborate and beryllium oxide it is true that a two-step evaluation by optically stimulated luminescence is possible. This is true in particular since the respective stimulation spectra and emission spectra of beryllium oxide and lithium tetraborate differ. Insofar the advantages disclosed in the named contexts apply here in analogy. As additional advantage it results that in the case of using beryllium oxide and lithium tetraborate an effective atomic number is achieved, which only has a slight deviation from the effective atomic number of human tissue, since this is the case already for effective atomic number of both components, beryllium oxide and lithium tetraborate, individually.

According to a further development of the first and/or second aspect of the invention it is envisaged that the shares in the converter material and the detector material in the composite material are chosen in such a way that an effective atomic number of between 6.1 and 8.1, preferably between 6.7 and 7.5 is rendered. This may be achieved by the share of beryllium oxide and/or lithium tetraborate in the composite material being chosen to be sufficiently high for compensating for a more significantly deviating effective atomic number of other components. In other words, an effective atomic number of the composite material in the interval of between 6.1 and 8.1, preferably between 6.7 and 7.5 can be ensured by the number of beryllium oxide and/or lithium tetraborate being sufficiently high. By choosing the effective atomic number in the named interval the initially named advantages for a corresponding atomic number close to the effective atomic number of human tissue can turn out to be particularly advantageous.

According to a further development it is envisaged that the particles of the converter material and/or the detector material have a grain size of less than 30 micrometers, preferably less than 10 micrometers. In other words, the converter material and/or the detector material each are present in particles the grain size of which is smaller than 30 micrometers, preferably smaller than 10 micrometers. As grain size therein for instance a diameter, a diagonal or maximum expansion of the corresponding particle in any random direction may be chosen. By a corresponding particle size, the advantages of the mixing of converter material and detector material in the material mixture can be further improved. In particular the distance the secondary radiation has to cover from the place of its generation, that is in the converter material until its detection in the detector material, can be further reduced.

According to a further development it is envisaged that the composite material has a flat surface as well as an expansion of between 0.2 millimeter and 0.5 millimeter, in particular an expansion of 0.3 millimeter, perpendicular to the surface. In other words, it may be envisaged that the composite material perpendicular to the flat surface has a thickness of between 0.2 millimeter and 0.5 millimeter, in particular a thickness of 0.3 millimeter. Preferably, the composite material is a cylindrical or square formation with a height of 0.2 millimeter to 0.5 millimeter, in particular with a height of 0.3 millimeter. In this way, on the one hand, a sufficient mechanical stability of the composite material is ensured. On the other hand, too large a thickness would be impedimental in a later evaluation by optically stimulated luminescence, which requires a transillumination of the entire detector with stimulation light. Typically, detectors are only partially transparent. The named thickness has turned out to be an advantageous compromise between evaluability and mechanical stability.

According to a further development it is envisaged that the converter material and the detector material are joined by burning or sintering into the composite material. In other words, the converter material and the detector material are brought together as loose particles in the material mixture. Subsequently, the composite material is joined by the burning or sintering. In particular the burning or sintering is so-called hot isostatic pressing. In this way a good mixing of converter material and detector material as well as a high rigidity of the composite material can be ensured.

A further aspect of the present invention relates to a dosimeter containing the composite material according to the invention as detector unit in at least one implementation. In particular the dosimeter has at least one detector unit with the composite material according to the invention and a further detector unit without converter material. By doing without the converter material the additional composite material should not be sensitive to free neutrons as it is the case e.g., with beryllium oxide. In this way the further composite material may serve for determining a reference value for a photon radiation which, in parallel to the incident neutrons, is absorbed by the dosimeter. The detector unit with the composite material according to the invention detects both the neutron radiation as well as the photon radiation. The further detector unit detects the photon radiation, not, though, the neutron radiation. In the later evaluation for both detector units a respective dose value can be determined. Subsequently, the photon radiation can be determined, which in part is in fact also detected by the dosimeter according to the invention. By subtraction of the reference value, that is the photon dose, from the dose value from the detector unit with the composite material according to the invention the pure neutron dose can be determined.

A further aspect of the present invention relates to a method for capturing free neutrons, comprising the steps:
a. at least partially absorbing the neutrons by a composite material, in which a converter material and a detector material each are present in a plurality of particles in a material mixture,
b. generating a secondary radiation by a converter material as a consequence of a neutron capture, and
c. storing the secondary radiation quantity by a detector material from beryllium oxide, which is configured to release or quantify, respectively, the information in a later evaluation by optically stimulated luminescence.

A still further aspect of the present invention relates to a method for capturing free neutrons, comprising the steps:
a. at least partially absorbing the neutrons by the composite material, in which a converter material and a detector material each are present in a plurality of particles in a material mixture,
b. generating a secondary radiation by a converter material from lithium tetraborate as consequence of a neutron capture, and
c. storing the secondary radiation quantity by a detector material, which is configured to release or quantify, respectively, the information in a later evaluation by optically stimulated luminescence.

The composite material, the dosimeter, as well as the evaluation of the composite material by optically stimulated luminance were already set out. The present method for capturing free neutrons additionally can be further developed by the corresponding features, which thus equally apply to the method. The respective advantages then apply in analogy.

The evaluation of the composite material by optically stimulated luminescence were already set out in the context of the composite material. The present method for detecting free neutrons additionally can be further developed by the corresponding features. The respective advantages then apply in analogy.

The storing is affected in particular in remanent manner. As information, in particular the cumulated quantity of the secondary radiation, is stored or, respectively, a value proportionately hereto. The detector material is preferably configured to release the stored information in a later evaluation dose-proportionately in the form of luminescence light.

The evaluation of the composite material by optically stimulated luminescence was already set out in the context of the composite material. The present method for detecting free neutrons can additionally be further developed by the corresponding features. The respective advantages then apply in analogy.

The invention further relates to a method for detecting free neutrons with the aid of a composite material comprising the steps of the method according to the fourth aspect of the present invention and/or the fifth aspect of the present invention as well as the following additional steps for evaluation of the information:
a. illuminating the composite material with light of a first stimulation spectrum, wherein the stimulation spectrum is specifically suited for stimulation of beryllium oxide or lithium tetraborate, and
b. detecting the neutrons based on an emission spectrum, which is emitted by the composite material in response to the illumination with the stimulation spectrum, corresponding to a predetermined provision

The evaluation of the composite material by optically stimulated luminescence was already set out in the context of the composite material. The present method for detecting free neutrons can additionally be further developed by the corresponding features. The respective advantages then apply in analogy. The two method steps of illuminating and detecting are in particular performed simultaneously. In particular the stored information in the present method is released dose-proportionately in the form of luminescence light.

According to a further development of the method for detecting free neutrons it is envisaged that
a. the composite material contains beryllium oxide as detector material as well as lithium tetraborate as converter material, and
b. the illuminating of the composite material is affected with two different stimulation spectra, wherein a first one of the two stimulation spectra is suitable for beryllium oxide and the other one of the two stimulation spectra for lithium tetraborate is specifically suited for the excitation of the optically stimulated luminescence.

In other words, the method provides a double or two-step evaluation by optically stimulated luminance. In the course of the twofold evaluation the information stored in each case in the beryllium oxide as well as the lithium tetraborate is released in particular consecutively or simultaneously. In this connection the composite material is illuminated consecutively or simultaneously with two different stimulation spectra. The two different stimulation spectra each can be provided by monochromatic light of different wavelength. The respective stimulation spectra or the respective wavelengths of the monochromatic light can be chosen specifically for the beryllium oxide and the lithium tetraborate. In particular the first stimulation spectrum is chosen in such a way that by same exclusively the beryllium oxide is excited for optically stimulated luminescence and/or the second stimulation spectrum is chosen in such a way that by same exclusively the lithium tetraborate is excited for optically stimulated luminescence. The illuminating with the first stimulation spectrum and the second stimulation spectrum may be affected simultaneously, consecutively, or in a temporally overlapping manner. In response to the respective illumination the lithium tetraborate and the beryllium oxide release the respective stored information simultaneously, consecutively, or in a temporally overlapping manner. This is affected by the emitting of the respective material-specific emission spectrum by the beryllium oxide and the lithium tetraborate. The two emission spectra can be detected separately or differentiated from each other, respectively. A read out of the two materials may be affected by differentiation of the respective emission spectra independently of each other. Subsequently, the independently determined values can be combined. On the whole, this embodiment results in a double evaluation of the information and thus an independent detection of the free neutrons by the beryllium oxide and the lithium tetraborate. In this way a clearly raised accuracy can be ensured.

In the following the invention is explained in further detail based on drawings of concrete embodiments. The shown embodiments therein are to be understood merely in an exemplary way and do not limit the invention. The figures are described as follows.

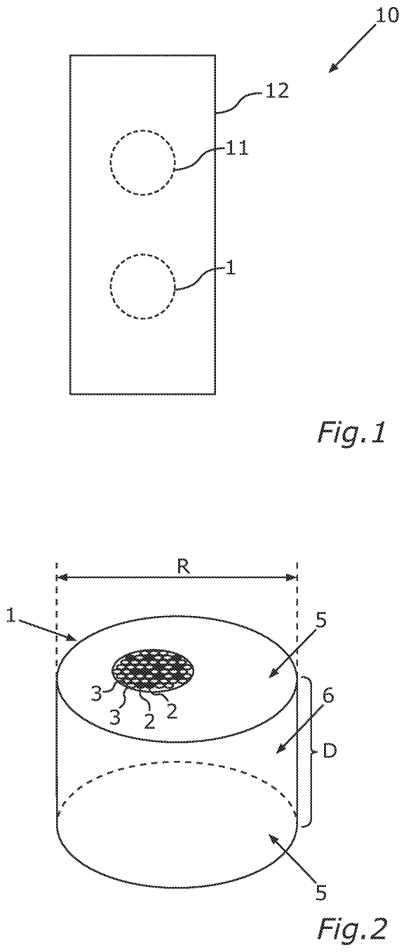

FIG. 1 depicts a dosimeter containing two detector units, in a schematic front view.

FIG. 2 depicts a composite material for a detector unit in a schematic perspective view.

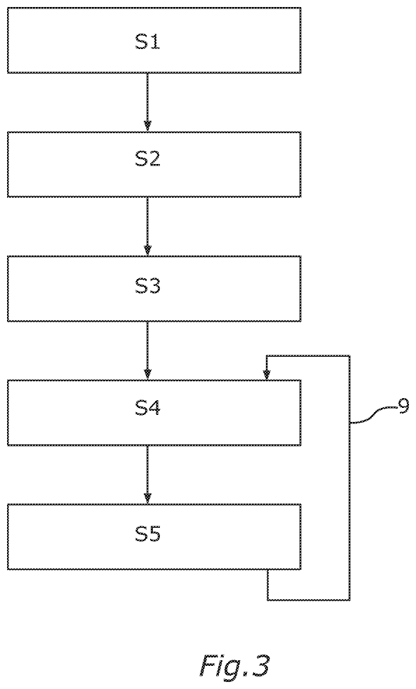

FIG. 3 depicts a flow diagram of an exemplary method for evaluating a neutron dose.

FIG. 1 shows a dosimeter 10, which comprises a housing 12. Within the housing 12 two detector units are arranged. A first one of the two detector units is provided by a composite material 1. The other one of the two detector units is referred to as further detector unit 11. Therein the dosimeter 10 is configured to capture neutron radiation, in particular so-called free neutrons and/or thermal neutrons. For capturing the free neutrons in particular the composite material 1 is configured. The further detector unit 11, in contrast, is configured to capture a photon radiation (gamma radiation, cosmic radiation, x-ray radiation, etc.) In the course of a later evaluation a reference value with regard to the captured photon radiation can be determined. By this reference value photon radiation captured by the composite material 1 can be subtracted so that as evaluation result solely the neutron dose detected by the composite material 1 remains. This later evaluation, however, in the following is yet to be discussed in more detail.

FIG. 2 shows the composite material 1 in a schematic perspective view. The composite material 1 in the present case exemplarily has a shape design similar to a tablet. In other words, the composite material 1 in the present case is merely exemplarily shaped in the form of a cylinder. The composite material 1 in the present case has two flat surfaces 5. In the present example the flat surfaces 5 moreover are parallel to each other. In the present example of a cylindrical shape design flat surfaces 5 are provided by the bottom and the top surface of the cylinder. Between the flat surfaces 5 in the present example extends the cylinder lateral surface 6. Perpendicular to one or both of the surfaces 5 the composite material has a thickness D. In the present example the flat surfaces 5 each are shaped to be circular, wherein a respective circle at the basis of the surfaces 5 has a diameter R.

The composite material 1 comprises a converter material 2, which is configured to generate a secondary radiation in response to a capture of free neutrons. A suitable converter material represents in particular chemical compounds containing the isotope ⁶Li. ⁶Li responds timely to the capture of free neutrons by a radioactive decay, in which short-range alpha radiation as well as a tritium particle are released. In the converter material accordingly, it is advantageously envisaged that the isotope ⁶Li compared to its natural frequency is enriched. Of course, any random materials may be considered as converter material 2 if they have significant capture cross sections for neutrons. Different materials in this connection can also generate different secondary radiation. However, frequently the source of the secondary radiation is a nuclear reaction caused by the neutron capture. In other words, the converter material 2 is advantageously characterized in that it contains atoms, which in response to a neutron capture radioactively decay whilst emitting the secondary radiation. In this connection it is to be ensured that the secondary radiation is generated in a period of time that is appropriate for the respective purpose of application. Advantageously, the converter material 2 or isotopes contained in the converter material 2, which are configured for capturing the neutrons and for generating the secondary radiation, have an as large as possible capture cross section for neutrons. The isotope ⁶Li for instance has a sufficiently large capture cross section for neutrons.

The composite material 1 further comprises a detector material 3, which is configured to store the quantity of the secondary radiation and have it determined in a later evaluation by optically stimulated luminescence. The detector material is in particular a material, which preserves the dose information by storing free charge carriers in stable energy levels. For instance, the traps which are capable of absorbing free charge carriers are energy levels which lie between valence band and conduction band of the detector material 3. A returning into the valence band or a raising into the conduction band starting from this energy level are not readily possible. This is the underlying principle to the fact that the electron is trapped on the corresponding energy level and can only be freed by further supply of energy. In the course of the later evaluation by optically stimulated luminescence by a corresponding energy supply the electron can be raised to an even higher energy level. When returning from this further raised energy level to the basic state or a different lower energy level, then a characteristic emission of light is generated, the wavelength of which corresponds to the released energy. This is explained in further detail in the following.

For application as part of the person dosimetry in the present case it is envisaged that the composite material 1 has an effective atomic number, which is very similar to the effective atomic number of human tissue. In this way measurement results, which are obtained by the composite material 1, to a considerable degree can be transferred to the human body or to a person wearing the dosimeter 10 on the body for monitoring of the exposition to radiation. In other words, by such a composite material 1 results relating to person dosimetry can be obtained, which in comparison with the prior art are improved. For instance, an effective atomic number in the composite material 1 of between 6.1 and 8.1, preferably of between 6.7 and 7.5 may be envisaged.

In order to obtain an effective atomic number, which complies with the above-named requirements, it may for instance be envisaged that the detector material 3 is formed from beryllium oxide. The effective atomic number of beryllium oxide (BeO) in good approximation (effective atomic number is 7.1) is equivalent to the effective atomic number of body tissue. Thus, radiation transport in the beryllium oxide takes place under similar conditions as in the human body. The composite material 1 thus can be used without additional filter in order to capture a dose over a wider energy range.

Beryllium oxide moreover is characterized in that a so-called fading, that is the loss of dose information over time, can be virtually neglected. Moreover, a typical detector sensitivity of beryllium oxide is high enough for reproducible measurements to be possible up into the dose range of few microsievert. Beryllium oxide in significant amounts are employed for applications as good thermally conductive insulator for example in ignition plugs and therefore are readily available as starting material also for an application in the dosimetry. As ceramic material beryllium oxide is chemically and mechanically very stable and not hygroscopic. Beryllium oxide has a sensitivity to incident photon radiation (x-ray, gamma) as well as electrons (beta radiation) and helium nuclei (alpha radiation) as far as these particles can enter the beryllium oxide, that is in the present case the detector material 3. In the pure form the detector material 3, that is in the present case the beryllium oxide, however, has no or only a minor sensitivity to the radiation with neutrons (thermal or high energy). For this reason, the admixing of the converter material 2 is envisaged in order to generate the secondary radiation, which then in turn is detectable with the aid of the detector material 3.

Another possibility to approximate the effective atomic number to the effective atomic number of human tissue consists in forming the converter material 2 from lithium tetraborate. Lithium tetraborate (Li₂B₄O₇) may even contain two possible isotopes with a high capture cross section for neutrons, namely ⁶Li and ¹⁰B. Lithium tetraborate with regard to its effective atomic number is equivalent in terms of tissue to human body tissue. In order to improve the efficiency as converter material 2, the isotope ⁶Li may be enriched compared to other lithium isotopes and/or the isotope ¹⁰B compared to other boron isotopes.

This means that according to a first embodiment it may be envisaged to combine in the composite material 1 lithium tetraborate as converter material 2 with any random detector material 3, which facilitates optically stimulated luminescence. Alternatively, according to a second embodiment it is possible to combine beryllium oxide as detector material 3 with any random converter material 2 which is configured to generate a secondary radiation in response to the incidence of free neutrons. Therein, in each case it is to be seen to it that the shares of the beryllium oxide or the lithium tetraborate in the composite material are sufficiently large to shift the mean effective atomic number of the entire composite material 1 to a value deviating from the effective atomic numbers of human tissue to maximally a predetermined extent. For instance, the share in beryllium oxide or the share in lithium tetraborate in the composite material 1 is to be chosen high enough for an effective atomic number for the entire composite material 1 of between 6.1 and 8.1, preferably of between 6.7 and 7.5, to be rendered.

Generally, it is envisaged that the composite material 1 in each case comprises at least 10 percent of the converter material 2 and of the detector material 3. Advantageously, the detector material 3 comprises a share of more than 10 percent, for instance at least 20 percent, at least 30 percent, at least 50 percent, or at least 70 percent, in order to sustain in the later evaluation a sufficient intensity of the luminescence. In this way, on the one hand, a sufficient conversion of the neutrons and, on the other and, a sufficient storage of the secondary radiation is ensured.

An effective atomic number having a particularly high tissue equivalence then invariably is rendered if as converter material 2 lithium tetraborate and as detector material 3 beryllium oxide is used. According to a third embodiment it may thus be envisaged that both the converter material 2 as well as the detector material 3 have tissue equivalence with regard to the respective effective atomic number. In this case it is in particular possible to combine lithium tetraborate as converter material 2 with beryllium oxide as detector material 3 in the composite material 2.

Due to the fact that the composite material comprises beryllium oxide as detector material 3 and lithium tetraborate as converter material 2, the effective atomic number irrespectively of the respective weight portions is equivalent to the effective atomic number of human tissue and the volume share of the converter material freely selectable. As a matter of course, these advantages are also entailed if a different converter material 2 than lithium tetraborate and/or a different detector material 3 than beryllium oxide with a comparable effective atomic number are chosen.

According to a fourth embodiment it may be envisaged that the lithium tetraborate is employed both as converter material 2 and as detector material 3. This is due to the fact that lithium tetraborate equally facilitates the storing of information with regard to the secondary radiation as well as a later release of this information by optically stimulated luminescence. In other words, the lithium tetraborate in an application as converter material 2 and detector material 3, on the one hand, can generate the secondary radiation in response to the capturing of the neutrons and equally store an information with regard to the secondary radiation itself. In this connection the capturing of the neutrons as well as the generating of the secondary radiation is affected in particular by the atoms contained in the lithium tetraborate ⁶Li and/or ¹⁰B. The storing of information with regard to the secondary radiation, in contrast, is affected substantially by the chemical compound of the lithium tetraborate.

Due to the in parts low range of the secondary radiation and/or in order to avoid an attenuation of the secondary radiation on its path from the converter material 2 to the detector material 3, in the present case it is envisaged that the converter material 2 and the detector material 3 each are present in a plurality of particles, which are jointly present in the composite material 1 as material mixture. This is schematically shown in FIG. 2. In other words, the converter material and/or the detector material 3 each are present in a plurality of particles. The respective particles of the converter material 2 and the detector material 3 are mixed in with each other in the material mixture 1. In this way the distance to be covered by the secondary radiation from the converter material 2 to the detector material 3 can be minimized. This applies in particular if the respective particles in which the converter material 2 and/or the detector material 3 is present have a grain size of less than 30 micrometer, in particular less than 10 micrometer.

The composite material 1 can for instance be produced by pressing, burning, and/or sintering. In the present embodiment the composite material 1 is produced by hot isostatic compressing. The starting material for this are the converter material 2 as well as the detector material 3 each in powder form. As described in the above, the respective grain sizes of the particles are a possible degree of freedom in manufacture. The composite material 1 moreover optionally may contain a binding agent to improve the cohesion of the individual particles. After the hot isostatic compressing by burning at a high temperature a stable ceramic can be produced. Degrees of freedom in order to optimize the manufacture therein consist in temperatures, temperature profiles, and the burning time. Burning temperatures therein may for instance be at about 1500 degree Celsius. Binding agents possibly employed in the pressing may decompose at least partially during burning at such temperatures. By the compressing and the subsequent burning a stable ceramic is produced. The composite material 1 is mechanically stable and inert. In particular, the composite material 1 is very stable against abrasion. Moreover, a composite material 1 is produced that is chemically very stable. Also, the composite material 1 after corresponding treatment is not hygroscopic, that is it does not attract water.

Finally, FIG. 3 shows a method for detecting free neutrons. The method for detecting free neutrons comprising the steps S1 to S5 in this connection contains a method for capturing free neutrons comprising the steps S1 to S3. In a first step S1 the composite material 1 is exposed to free neutrons. Therein at least part of the free neutrons is absorbed by the composite material 1.

In a step S2 by the converter material 2 a secondary radiation is generated in response to the presence of neutrons. In particular the neutrons are captured by the converter material 2 at least partially whilst generating the secondary radiation. In particular the neutrons are captured by the converter material 2 at least partially whilst generating the secondary radiation. In a further step S3 an information with regard to the secondary radiation is stored by the detector material 3. The detector material 3 further is configured to release the information with regard to the secondary radiation again at a later evaluation by optically stimulated luminescence.

It is to be noticed that the steps S1, S2, and S3 in reality commonly are executed to be temporally overlapping or even simultaneously.

In the performance of the method, it may be envisaged that either the converter material is formed from lithium tetraborate or the detector material is formed from beryllium oxide. Alternatively, it may be envisaged that the converter material 2 is formed from lithium tetraborate and at the same time the detector material 3 is formed from beryllium oxide. According to a further alternative it may be envisaged that both the converter material 2 and the detector material 3 are formed from lithium tetraborate.

The later evaluation may substantially be given by the further steps S4 and S5. In a step S4 the composite material 1 is illuminated with light of a stimulation spectrum. Therein the stimulation spectrum for the detector material 3, that is beryllium oxide or lithium tetraborate, is specifically suited for stimulation. In particular the stimulation spectrum is at least substantially monochromatic light, wherein a wavelength of the at least substantially monochromatic light is specific for the detector material 3, that is in particular beryllium oxide or lithium tetraborate. Specific means in particular that an energy of photons of the stimulation spectrum is sufficient to free electrons from the traps. In another step S5 in particular simultaneously with step S4 an emission spectrum is detected, which is emitted by the composite material 1, in particular the detector material 3, in response to the illumination with the stimulation spectrum. Therein according to a predetermined provision an intensity of the neutrons can be derived from the intensity of the emission spectrum. In particular a neutron dose is determined from the number of photons of the emission spectrum. For instance, the determined neutron dose according to the predetermined provision may be proportional to the number of detected photons of the emission spectrum. The photons of the emission spectrum are in particular monochromatic light of a second wavelength. The second wavelength is in particular specific for the detector material 3, in particular beryllium oxide or lithium tetraborate.

The steps S4 and S5 are in particular performed simultaneously. This may be due to the fact that the detector material reacts at least nearly instantaneously with the emission of the emission spectrum in response to the stimulation with the stimulation spectrum. In order not to lose any dose information, however, it is also necessary to perform the detecting according to step S5 during the entire duration of the emission of the emission spectrum.

Finally, as part of the present method a loop 9 may be performed so that the steps of illuminating the composite material and the detecting of the neutrons, that is the steps S4 and S5, are multiply performed. Therein it is in particular envisaged that the illuminating of the composite material is affected consecutively or simultaneously with the two different stimulation spectra. This is reasonable in particular if the converter material 2 is formed from lithium tetraborate and the detector material 3 from beryllium oxide. This is because, as already described in the above, in this case two different materials, which facilitate an evaluation by optically stimulated luminescence, are present in the composite material 1. Accordingly, it may be envisaged that in the step S4 the composite material 1 is consecutively or simultaneously illuminated with the two different stimulation spectra, wherein a first one of the two different stimulation spectra is specific for beryllium oxide and the other one of the two stimulation spectra is specific for lithium tetraborate. Analogously, then two different emission spectra are detected, wherein a first one of the emission spectra may be specific for beryllium oxide and the other one of the two emission spectra for lithium tetraborate. Since both the stimulation spectra and the emission spectra each may differ from each other, it is possible to perform the illuminating with the two stimulation spectra as well as the detecting of the two emission spectra simultaneously. By the respectively different wavelengths a mutual influencing can possibly be excluded. Alternatively, it is possible that the illuminating of the composite material 1 with the two different stimulation spectra is carried out consecutively. Accordingly, in this case also the detecting of the two stimulation spectra is executed consecutively. The illuminating with the first stimulation spectrum and the detecting of the first emission spectrum is affected simultaneously. Analogously, the illuminating with the second stimulation spectrum and the detecting of the second emission spectrum is affected simultaneously.

As part of the evaluation also the reference value with regard to the captured photon radiation may be determined. The reference value is determined by the further detector unit 11. The further detector unit 11 may be modeled on the composite material 1, however, the further detector unit 11 does not comprise any converter material 2. Thus, the further detector unit 11 has no significant or only a very low sensitivity to neutron radiation. For instance, the sensitivity to neutron radiation of the further detector unit 11 compared with the composite material 1 is lower at least by the factor 10 or 100. For instance, the further detector unit 11 captures exclusively ionizing radiation, in particular the photon radiation. By the reference value then photon radiation captured by the composite material 1 can be subtracted so that as evaluation result solely the neutrons detected by the composite material 1 are obtained.

### LIST OF REFERENCE SIGNS

1 composite material
2 converter material
3 detector material
5 surfaces
6 cylinder lateral surface
9 loop
10 dosimeter
11 composite material
12 housing
D thickness
R diameter
S1 method step
S2 method step
S3 method step
S4 method step
S5 method step

1.-15. (canceled)

16. A composite material for detecting free neutrons, comprising:
a converter material that is configured as a consequence of a neutron capture to generate a secondary radiation; and
a detector material that is configured to store an information relating to a quantity of the secondary radiation and to release it again in a later evaluation by optically stimulated luminance,
wherein the converter material and the detector material each are present in a plurality of particles, which jointly are present in the composite material as material mixture, and
wherein the detector material is formed from beryllium oxide.

17. A composite material for detecting free neutrons, comprising:
a converter material that is configured as a consequence of a neutron capture to generate a secondary radiation; and
a detector material that is configured to store an information relating to a quantity of the secondary radiation and to release it again in a later evaluation by optically stimulated luminescence,
wherein the converter material and the detector material each are present in a plurality of particles, which are jointly present in the composite material as material mixture, and
wherein the converter material is formed from lithium tetraborate.

18. The composite material according to claim 17, wherein in the lithium tetraborate the isotopes 6Li and/or 10B compared to their natural frequency are enriched.

19. The composite material according to claim 17, wherein the detector material is formed from a different material than lithium tetraborate.

20. The composite material according to claim 19, wherein the detector material is formed from beryllium oxide.

21. The composite material according to claim 17, wherein the detector material is formed from lithium tetraborate.

22. The composite material according to claim 17, wherein shares of the converter material and the detector material in the composite material are chosen in such a way that an effective atomic number of between 6.1 and 8.1, or between 6.7 and 7.5 is rendered.

23. The composite material according to claim 17, wherein the particles of the converter material and/or the detector material have a grain size of less than 30 micrometers, or of less than 10 micrometers.

24. The composite material according to claim 17, wherein the composite material has a flat surface as well as an expansion of between 0.2 millimeter and 0.5 millimeter, or an expansion of 0.3 millimeter, perpendicular to the flat surface.

25. The composite material according to claim 17, wherein the converter material and the detector material are joined by burning or sintering into the composite material.

26. A dosimeter comprising:
a composite material according to claim 17.

27. A method for capturing free neutrons, comprising:
at least partially absorbing the neutrons by a composite material having a converter material and a detector material, wherein the converter material and the detector material each are present in a plurality of particles in a material mixture, and the detector material is formed from beryllium oxide;
generating a secondary radiation by the converter material as a consequence of a capture of the neutrons; and
storing an information relating to a quantity of the secondary radiation by the detector material of beryllium oxide, which is configured to release the information again in a later evaluation by optically stimulated luminescence.

28. A method for detecting free neutrons with the aid of a composite material comprising the steps of the method according to claim 27, and further comprising evaluating the information by:
illuminating the composite material with light of a stimulation spectrum, wherein the stimulation spectrum is specific for at least one of beryllium oxide or lithium tetraborate, and
detecting the neutrons based on an emission spectrum, which is emitted by the composite material in response to the illumination with the stimulation spectrum, corresponding to a predetermined provision.

29. The method according to claim 28, wherein
the composite material contains lithium tetraborate as the converter material, and
the illuminating of the composite material is affected with two different stimulation spectra, wherein one of the two stimulation spectra is specific for beryllium oxide and the other of the two stimulation spectra for lithium tetraborate.

30. A method for capturing free neutrons, comprising:
at least partially absorbing the neutrons by a composite material having a converter material and a detector material, wherein the converter material and the detector material each are present in a plurality of particles in a material mixture, and the converter material is formed from lithium tetraborate;
generating a secondary radiation by the converter material from lithium tetraborate in response to a presence of neutrons; and
storing an information relating to a quantity of the secondary radiation by the detector material, which is configured to release the information again in a later evaluation by optically stimulated luminescence.

31. A method for detecting free neutrons with the aid of a composite material comprising the steps of the method according to claim 30, and further comprising evaluating the information by:
illuminating the composite material with light of a stimulation spectrum, wherein the stimulation spectrum is specific for at least one of beryllium oxide or lithium tetraborate, and
detecting the neutrons based on an emission spectrum, which is emitted by the composite material in response to the illumination with the stimulation spectrum, corresponding to a predetermined provision.

32. The method according to claim 31, wherein
the composite material contains beryllium oxide as the detector, and
the illuminating of the composite material is affected with two different stimulation spectra, wherein one of the two stimulation spectra is specific for beryllium oxide and the other of the two stimulation spectra for lithium tetraborate.
